(19) United States
(12) Plant Patent Application Publication
(10) Pub. No.: US 2016/0073561 P1
(43) Pub. Date: Mar. 10, 2016

(54) Vaccinium plant named 'WINTER BELL'

(50) ;

(71) Applicant: David Weber, Winter Haven, FL (US)

(72) Inventor: David Weber, Winter Haven, FL (US)

(21) Appl. No.: 14/121,437

(22) Filed: Sep. 8, 2014

(51) Int. Cl. A01H 5/00

(52) U.S. Cl. PLT/156

(57) ABSTRACT

A new and distinct Vaccinium spp. cultivar named ‘WINTER BELL’ is disclosed, characterized by a semi-upright plant habit, vigorous growth and semi-deciduous habit in Florida. The new variety shows tolerance for a wetter soil conditions. Fruit yield is exceptionally high, with fruit ripening early, with first harvest in mid to late March. Fruit is somewhat bell-shaped with good size and an attractive waxy coating. The new variety is a Vaccinium, and is normally used as an ornamental garden or landscape plant.

### LATIN NAME OF THE GENUS AND SPECIES

Vaccinium spp.

### VARIETY DENOMINATION

‘WINTER BELL’

### BACKGROUND OF THE INVENTION

The new cultivar was discovered as the product of a breeding program conducted in Winter Haven, Fla., by the breeder David Weber. ‘Winter Bell’ was selected from open pollinated seedlings of the undistributed, unpatented variety referred to by code FL 95-174 which were planted in Winter Haven, Fla. in June, 2008. The pollen parent is unknown. Due to its earliness and fruit quality ‘Winter Bell’ was selected for yield evaluation in 2012. ‘Winter Bell’ was evaluated as a single plant test against other commercial varieties in 2012 and 2013 and planted in a confidential, controlled field test plot in June, 2013.

‘Winter Bell’ has been asexually propagated over numerous generations by softwood cuttings in Winter Haven, Fla., and all propagated material has retained the vegetative and fruit characteristics of the mother plant. First asexual propagation was performed June 2012, in Winter Haven, Fla. at a commercial nursery.

Asexual reproduction of the new cultivar ‘WINTER BELL’ by vegetative cuttings was performed at the nursery in Hawthorne, Fla. in September of 2007. Vegetative reproduction by cuttings has produced multiple generations, and has shown that the unique features of this cultivar are stable and reproduced true to type through successive generations.

### SUMMARY OF THE INVENTION

The cultivar ‘WINTER BELL’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘WINTER BELL.’ These characteristics in combination distinguish ‘WINTER BELL’ as a new and distinct Vaccinium spp. cultivar:

1. Exceptionally high fruit yield

2. Early fruit ripening, first harvest in mid to late March

3. Vigorous growth in native spodic soils

4. Excellent fruit bloom(attractive waxy coating)

5. Excellent fruit size

6. Excellent fruit scar

7. Resistance to common root diseases, tolerating wetter soils.

8. Semi-evergreen to evergreen habit under Florida conditions.

9. Slight bell shape to fruit.

10. Semi-upright habit

11. Fruit of average firmness

### PARENT COMPARISON

Plants of the new cultivar ‘WINTER BELL’ are similar to plants of the parent variety in most horticultural characteristics. However, plants of the new cultivar ‘WINTER BELL’ differ from FL 95-174 in the following:

1. ‘Winter Bell’ has larger leaves

2. ‘Winter Bell’ produces larger fruit

3. Plants of ‘Winter Bell’ are more upright with less lateral branching.

4. Plants of ‘Winter Bell’ are grow more vigorously.

5. Fruit of ‘Winter Bell’ has a different shape.

6. Fruit occurs in loose clusters on ‘Winter Bell’, whereas fruit occurs on the parent variety more singularly along stem internodes.

7. Fruit pedicels of ‘Winter Bell’ are longer.

The pollen parent is unknown.

### COMMERCIAL COMPARISON

‘WINTER BELL’ can be compared to the commercial variety Vaccinium ‘Jewel’ U.S. Plant Pat. No. 11,807. Plants of the new variety are similar to ‘Jewel’ in most horticultural characteristics, however, ‘WINTER BELL’ differs in the following:

1. Earlier ripening than Jewel. In testing conducted during 2012, by March 20, Winter Bell produced 2.9 pounds of fruit whereas ‘Jewel’ produced had produced 0.28 pounds of fruit.

2. Plants of ‘Winter Bell’ are grow more vigorously.

3. Highly waxy leaves of ‘Winter Bell’ are more durable to physical stress.

4. Plants of ‘Winter Bell’ yield more fruit

5. Fruit of ‘Winter Bell’ has a different shape.

6. Leaves are more persistent and rust resistant than foliage of ‘Jewel’

‘WINTER BELL’ can be compared to the commercial variety Vaccinium ‘Emerald’ U.S. Plant Pat. No. 12,165. Plants of the new variety are similar to ‘Emerald’ in most horticultural characteristics, however, ‘WINTER BELL’ differs in the following:

1. Earlier ripening than Emerald. In testing conducted during 2012, by March 20, Winter Bell produced 2.9 pounds of fruit whereas ‘Emerald’ had no ripe fruit on this date. The subsequent measurement, March 27, resulted in 4.7 pounds total harvested to date from ‘Winter Bell’ and 2.9 pounds harvested to date from ‘Emerald’.

2. Plant habit of ‘Winter Bell’ is more upright

3. Plants of ‘Winter Bell’ are more tolerant of wet soils

4. Leaves of ‘Winter Bell’ are more waxy.

5. Stronger waxy coating of fruit, giving an attractive, cloudier blue coloration.

### BRIEF DESCRIPTION OF THE PHOTOGRAPHS

The accompanying photograph illustrate the following:

FIG. 1 illustrates in full color a typical plant of ‘WINTER BELL’ grown outdoors in Winter Haven, Fla. This plant is approximately 9 months old.

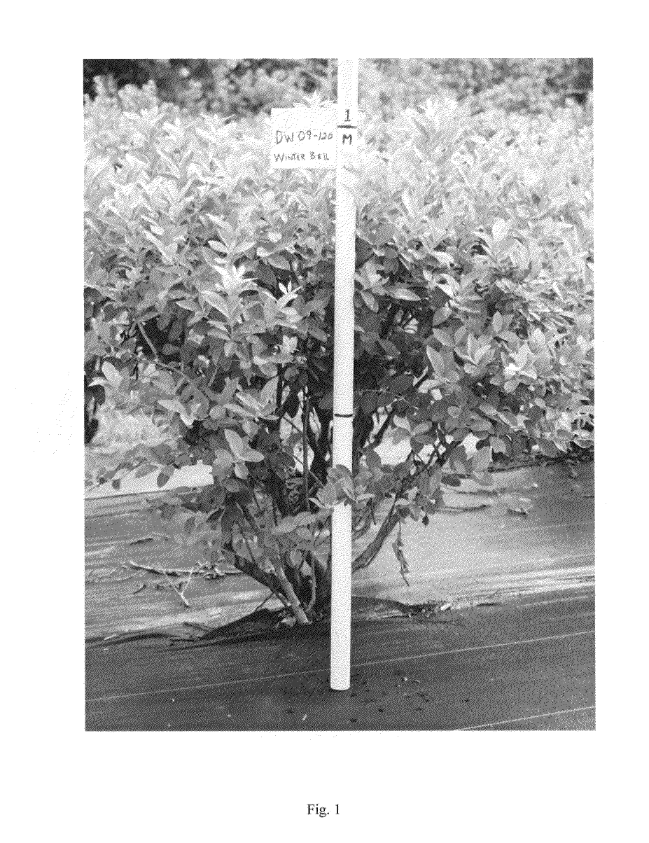

FIG. 2 illustrates in full color typical immature fruit and mature flowers of ‘Winter Bell’.

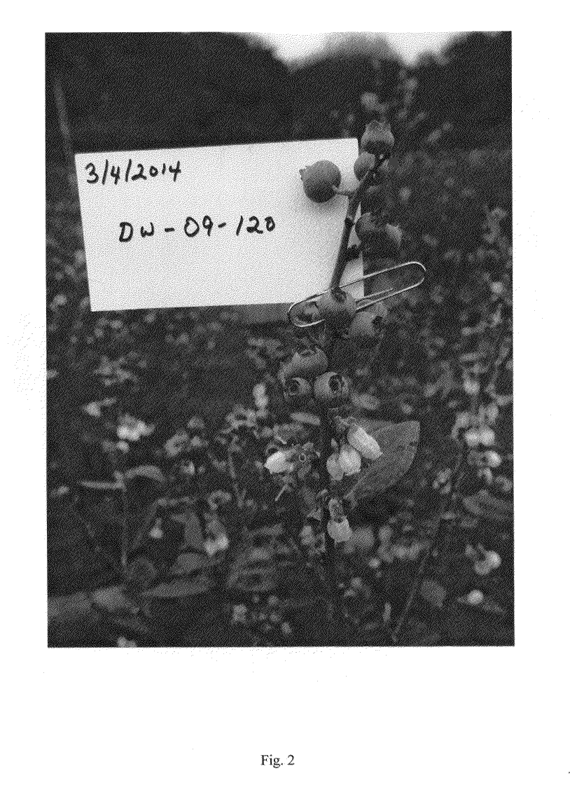

FIG. 3 illustrates in full color typical mature and immature fruit on a plant of ‘WINTER BELL’.

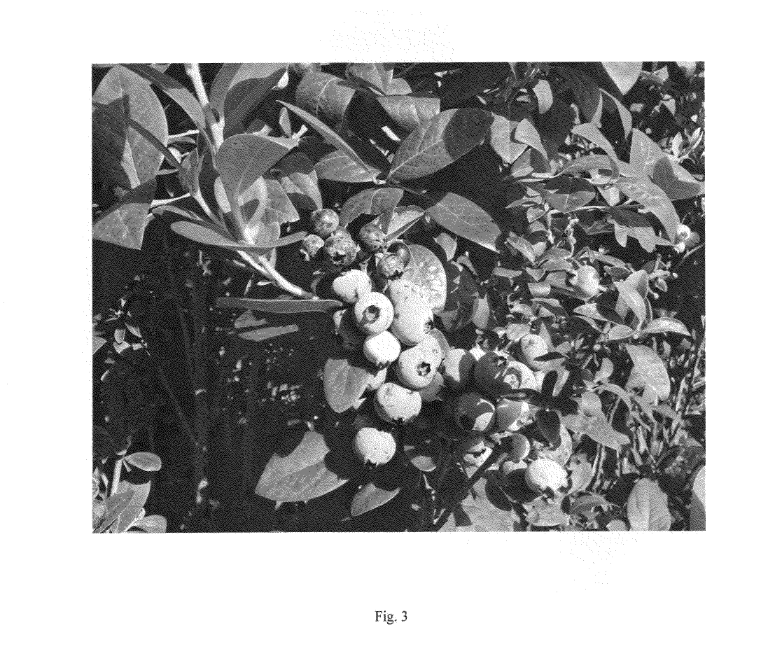

FIG. 4 illustrates in full color typical fruits of ‘WINTER BELL’, referred to in the photo by the breeder code 09-120(DW) and typical fruits of ‘Emerald’.

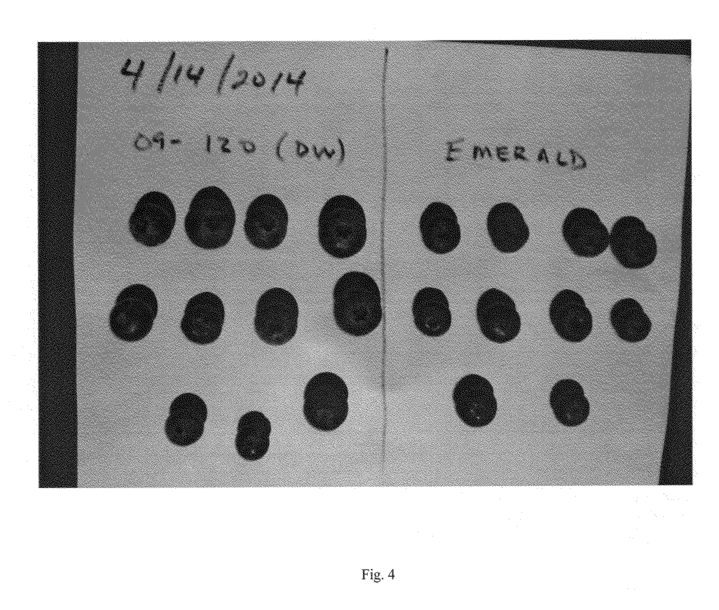

FIG. 5 illustrates in full color a typical mature plant of ‘WINTER BELL’ with immature fruit set.

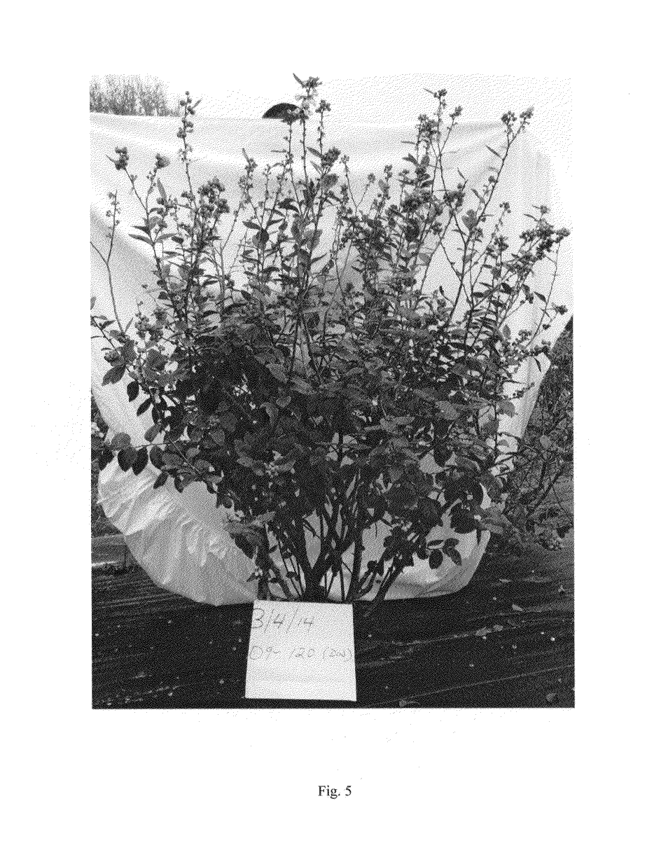

The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Colour Chart 2007 except where general terms of ordinary dictionary significance are used. The following observations and measurements describe ‘WINTER BELL’ plants grown outdoors in Winter Haven, Fla. Year round temperature ranged from approximately 60° to 90° F., during the day and 50° to 70° F., at night with naturally high light conditions. Measurements and numerical values represent averages of typical flowering types. No chemical or photoperiodic treatments were given.
Botanical classification: Vaccinium hybrid ‘WINTER BELL’
Age of plant described: Approximately 9 months old, planted the ground.
Propagation: Typically from semi-hardwood juvenile cuttings.
Plant:
Growth habit: Upright, semi-deciduous woody perennial shrub.
Height: Approximately 1 meter.
Plant spread: Approximately 70 cm.
Growth rate: Vigorous.
Branching characteristics: Free branching.
Texture of lateral branches: Smooth.
Color of lateral branches: Near RHS Green 143C
Aspect: Straight, occurring at approximately 15 to 45 degree angles
Strength of lateral branches: Very strong, very flexible.
Foliage:
Leaf:
   Arrangement.—Alternate.
   Average length.—Approximately 7 cm.
   Average width.—Approximately 4 cm.
   Shape of blade.—Broad elliptic.
   Apex.—Acute.
   Base.—Obtuse.
   Attachment.—Stalked.
   Margin.—Entire.
   Texture of top surface.—Highly waxy, glabrous.
   Texture of bottom surface.—Highly waxy, glabrous.
   Leaf internode length.—Approximate range 0.9 cm to 1.5 cm.
   Color.—Young foliage upper side: Near RHS Green 138A Young foliage under side: Near RHS Green 138C Mature foliage upper side: Near RHS Green 137A, but much darker. Mature foliage under side: Near RHS Green 138B.
   Venation.—Type: Pinnate. Venation color upperside: Near RHS Green 143B Venation color underside: Near RHS Green 143A.
   Petiole.—Length: Approximately 0.2 cm Diameter: Approximately 0.2 cm Texture: Smooth. Color upperside: Near RHS Yellow-Green 144C. Color underside: Near RHS Green 143B.
Flower:
Natural flowering season: 50% bloom open on Feb. 15, 2014, in Winter Haven, Fla.
Inflorescence and flower type and habit: Urceolate flowerw arrange in small, alternating racemes.
Quantity of flowers per raceme: Approximately 4 to 15 flowers per raceme, variable.
Diameter of raceme: Average approximately 4 to 9 cm
Depth of raceme: Average approximately 5 to 12 cm
Fragrance: Not observed.
Bud:
   Shape.—Obovate.
   Length.—Approximately 1.0 cm.
   Diameter.—Approximately 0.8 cm.
   Color.—Near RHS Yellow-White 158A.
Flower size:
   Diameter.—Approximately 0.8 cm.
   Length.—Approximately 1.1 cm.
   Corolla tube length.—Approximately 0.9 cm.
Petals:
   Quantity.—5.
   Arrangement.—Fused.
   Length.—Approximately 0.8 cm.
   Width.—Approximately 0.3 cm.
   Shape.—Completely fused, no individual shape.
   Apex.—Acute.
   Base.—Fused.
   Margin.—Entire.
   Texture, upper and lower surfaces.—Shiny.
   Color.—When opening, outer surface: Near RHS Red-Purple 62D When opening, inner surface: Near RHS Red 56C Fully opened, outer surface: Near RHS Red 56D Fully opened, inner surface: Near RHS Red 56D.
Calyx:
   Sepal quantity.—5.
   Arrangement.—Fused.
   Length.—Approximately 0.5 cm.
   Width.—Approximately 0.2 cm.
   Shape.—Deltoid.
   Apex.—Acute.
   Base.—Fused.
   Margin.—Entire.
   Texture, upper and lower surfaces.—Shiny.
   Color.—When opening, outer surface: Near RHS Red 54B When opening, inner surface: Near RHS Red 54B.
Pedicels:
   Length.—Approximately 0.3 cm.
   Diameter.—Approximately 0.1 cm.
   Strength.—Weak to moderate.
   Texture.—Shiny.
   Color.—Near RHS Greyed-Purple 186D.
Peduncles:
   Length.—Approximately 2 cm.
   Diameter.—Approximately 0.8 cm.
   Strength.—Moderate.
   Texture.—Shiny.
   Color.—Near RHS Greyed-Purple 183C.
Reproductive organs:
Stamens:
   Number.—Typically 10.
   Color.—Near RHS Yellow 7C.
   Length.—Approximately 0.3 cm.
Pistil:
   Number.—1.
   Length.—Approximately 0.9 cm.
   Style.—Length: Approximately 0.5 cm. Color: Near RHS Yellow-Green 145B.
   Stigma.—Shape: Narrowly conical. Color: Near RHS Yellow-Green 153D Ovary Color: Near RHS Yellow-Green 145A.
Fruit:
   Seasonal timing.—First fruit harvested typically mid-March.
   Color.—Immature: First near Yellow-Green 145C, changing to Blue 100C, Fully ripe: Colored close to Blue 103A, but much darker, waxy blush coloration near Blue 106D. Ripe internal coloration: Near Yellow-Green 145D.
   Diameter.—Average 1.4 cm.
   Diameter.—Average 1.4 cm.
Other characteristics:
Disease resistance: Higher than average resistance to normal foliar diseases normal to Vaccinium, have been observed in this variety. Resistance to disease found in wet soil conditions also observed.
Pest resistance: Neither resistance nor susceptibility to the normal pests of Vaccinium.
Drought tolerance and temperature tolerance: Low temperature tolerance and drought tolerance have not been tested.

What is claimed is:

1. A new and distinct cultivar of Vaccinium plant named ‘WINTER BELL’ as herein illustrated and described.
